(19) United States
(12) Patent Application Publication
(10) Pub. No.: US 2002/0031554 A1
(43) Pub. Date: Mar. 14, 2002

(54) FOOD SUPPLEMENT COMPOSITION BASED ON DEHYDRATED CRAB MEAT

(76) Inventor: NEVILLE TREVOR WITTENBAKER, BAYSWATER (AU)

Correspondence Address: GREGORY P LAPOINTE, BACHMAN & LAPOINTE P.C., 900 CHAPEL STREET, SUITE 1201, NEW HAVEN, CT, 06510, US

(21) Appl. No.: 09/485,448

(22) PCT Filed: Aug. 12, 1998
(86) PCT No.: PCT/AU98/00633

(30) Foreign Application Priority Data
Aug. 12, 1997 (AU) PO 8512

(51) Int. Cl. A61K 35/64; A23L 1/325

(52) U.S. Cl. 424/538; 426/643

(57) ABSTRACT

A food supplement composition which is derived from dehydrated crabs and which contains 150 to 250 g/kg of protein, 200,000 to 300,000 mg/kg of calcium, 10,000 to 15,000 mg/kg of phosphorus and 200 to 300 mg/kg of iron. The food supplement composition may be taken directly or in admixture with liquid or solid food or drink. It is beneficial for people with iron, calcium or protein deficiencies, and relieves the symptoms of asthma when taken by a person suffering from asthma.

### FIELD OF THE INVENTION

The present invention relates to a food supplement composition.

### BRIEF DESCRIPTION OF THE INVENTION

In accordance with one aspect of the present invention there is provided a food supplement composition which is derived from dehydrated crabs and which includes protein, calcium, phosphorus and iron.

### DESCRIPTION

The composition of the present invention is prepared from crabs preferably of the species Scylla serrata, which are more commonly known as mud crabs. Preferably, the mud crabs are black, dark brown or chestnut brown in colour, or light green, dark blue or purplish in colour.

Intestines and digestive organs of the crab are preferably removed before preparation of the composition of the present invention The remaining crab material may then be heated to drive off water. Preferably, the heating is done on an open fire with the crab material being held above the flame on a chemically inert utensil such as a stainless steel utensil. The fire is preferably a wood or charcoal fire. It will be appreciated that alternative heat sources such as gas or electric stoves, conventional convection, vacuum or microwave ovens may be used to dehydrate the crab material.

In this way the crab material may be dehydrated until substantially all of the water content thereof is driven off and the crab material has turned to a dark colour. The dehydrated crab material may then be ground up to form a powder.

The resultant composition includes protein, calcium, phosphorus and iron and is suitable for human consumption The composition may be taken as a food supplement, either directly in a dry powder form or in admixture with solid or liquid food or drink. The composition may be sold in capsule form, in a gel, in tablet form or in a liquid as a solution or a syrup. The composition is conveniently taken with honey. The composition may be taken in suitable doses several times a day.

Preferably, the composition of the present invention contains from 150 to 250 g/kg of protein, from 200,000 to 300,000 mg/kg of calcium, from 10,000 to 15,000 mg/kg of phosphorus and from 200 to 300 mg/kg of iron. The composition of the present invention is useful as a food supplement and is a good supplement for providing protein and essential body mineral requirements. For example, it is very useful for people with low levels of minerals such as people with iron deficiency or calcium deficiency. It is a useful dietary supplement for people on a high protein diet, or who suffer from protein deficiency.

The composition of the present invention when taken by a person suffering from asthma relieves the symptoms of the asthma, and is therefore good for the symptoms of asthma.

The present invention will now be illustrated by the following example.

### EXAMPLE

A sample of mud crab material with intestines removed was heated in a steel utensil over an open wood fire for 30 minutes. The crab material was dehydrated because almost all of the water content was driven off. Further, the crab material darkened in colour during the heating. The resultant residue was ground up to form a dark coloured powder. The powder was analysed and was found to have the composition set out in the following table.

As can be seen, in column 2 the recommended dose for this composition is 2 grams taken 3 times a day. At this level beneficial amounts of protein, calcium, phosphorous and iron are ingested daily by the person taking the powder. On the other hand the amounts of other ingredients such as lead, cadmium, mercury, selenium, zinc and copper is ingested -15 are well below the maximum amounts recommended in the Food Standards Code as shown by columns 2 and 3.

The recommended dose for children of the composition of this example is half the adult dose giving a total of 3 grams per day.

Private clinical patients suffering from chronic asthma or respiratory-related diseases show a reduction of symptoms with little or no adverse side effects.

| | | | 3. Food Standards |
|---|---|---|---|
| | | | Code Maximum |
| | | 2. Adult Dose | values for “Foods |
| | | 2 grams to be | not containing a |
| | | taken 3 | food not otherwise |
| ANALYSIS | 1. Results | times/day | standardised” |
| Nitrogen, N g/kg | 36.5 | 0.22 | — |
| Protein,N*6.25 g/kg | 228 | 1.368 | — |
| Fat g/kg | 8 | 0.05 | — |
| Calcium,Ca mg/kg | 270,000 | 1620 | — |
| Phosphorus, P mg/kg | 12,500 | 75 | — |
| Iron, Fe mg/kg | 270 | 1.6 | — |
| Lead, Pb mg/kg | 4 | 0.02 | 1.5 |
| Cadmium, Cd mg/kg | <0.5 | <0.003 | 0.05 |
| Mercury, Hg mg/kg | <0.5 | <0.003 | 0.03 |
| Selenium, Se mg/kg | <10 | <0.06 | 1.0 |
| Zinc, Zn mg/kg | 90 | 0.5 | 150 |
| Copper, Cu mg/kg | 12 | 0.07 | 10 |
| Carbohydrate, | 86 | 0.5 | — |
| total g/kg | | | |
| Carbohydrate, | negligible | negligible | — |
| sugars g/kg | | | |
| Sodium, Na g/kg | 20.3 | 0.12 | — |
| Potassium, K g/kg | 6.7 | 0.04 | — |

Modifications and variations as would be apparent to a skilled addressee are deemed to be within the scope of the present invention.

1. A food supplement composition characterised in that it is derived from dehydrated crabs from which digestive organs thereof have been removed and which includes protein, calcium, phosphorus and iron.

2. A food supplement composition according to claim 1, characterised in that the crabs are Scylla serrata.

3. A food supplement composition according to claim 1, characterised in that the crabs are mud crabs.

4. A food supplement composition according to any one of claims 1 to 3, characterised in that the crabs are black, dark brown, chestnut brown, light green, dark blue or purplish in colour.

5. A food supplement composition according to any one of the preceding claims, characterised in that it includes 150 to 250 g/kg or protein, 200,000 to 300,000 mg/kg of calcium, 10,000 to 15,000 mg/kg of phosphorus and 200 to 300 mg/kg of iron.

6. A process for the preparation of a food supplement composition according to any one of claims 1 to 5, characterised in that the process includes the following steps:
a) the crab is heated until the crab is dehydrated and has turned a dark colour; and
b) the crab is ground to a powder.

7. A process for the preparation of a food supplement composition according to claim 6, characterised in that digestive organs of the crab are removed before the crab is heated to dehydration.

8. A process for the preparation of a food supplement composition according to claims 6 or 7, characterised in that the crab is heated to dehydration in a chemically inert utensil.

9. A process for the preparation of a food supplement composition according to any one of claims 6 to 8, characterised in that a heat source used to heat the crab to dehydration is an open wood or charcoal fire, a gas or electric stove, a conventional convection, microwave or vacuum oven.

10. A pharmaceutical composition including the food supplement composition according to any one of claims 1 to 3, as active ingredient, optionally in admixture with a suitable carrier.

11. A pharmaceutical composition according to claim 10 in the form of a gel, a capsule, a tablet, a solution, or a syrup.

12. The use of a food supplement composition according to any one of the preceding claims, for the preparation of medicaments for the treatment of asthma.

13.The use of a food supplement according to any one of the preceding claims for the preparation of medicaments for the treatment of symptoms of respiratory- related diseases.

14. The use of a food supplement composition according to any one of the preceding claims, for the preparation of medicaments for the treatment of iron deficiency.

15. The use of a food supplement composition according to any one of the preceding claims, for the preparation of medicaments for the treatment of calcium deficiency.

16. The use of a food supplement composition according to any one of the preceding claims, for the preparation of medicaments for the treatment of protein deficiency.
